(19) United States
(12) Patent Application Publication
(10) Pub. No.: US 2008/0095843 A1
(43) Pub. Date: Apr. 24, 2008

(54) Controlled-release formulations

(76) Inventors: Siva Rama K. Nutalapati, Princeton, NJ (US); Kristin Arnold, Morrisville, PA (US); Ishari Piya, Lansdale, PA (US)

Correspondence Address: CANTOR COLBURN, LLP, 20 Church Street, 22nd Floor, Hartford, CT, 06103, US

(21) Appl. No.: 11/827,368

(22) Filed: Jul. 11, 2007

Related U.S. Application Data
(60) Provisional application No. 60/819,914, filed on Jul. 11, 2006.
(60) Provisional application No. 60/859,772, filed on Nov. 17, 2006.

(51) Int. Cl. A61K 31/137; A61K 9/42; A61P 11/00

(52) U.S. Cl. 424/465; 514/653

(57) ABSTRACT

Disclosed herein are controlled-release formulations which exhibit substantially zero-order release kinetics. The formulations include a core comprising a core active agent and a wax excipient substantially coated with an extended-release coating. The formulation optionally includes an immediate-release portion comprising an immediate-release active agent in the form of, for example, a coating disposed on at least a portion of the core. Further disclosed are fexofenadine/pseudoephedrine combination formulations, which exhibit substantially no food effect.

### CROSS REFERENCE TO RELATED APPLICATION

This application claims priority from U.S. Provisional Application Ser. Nos. 60/819,914 filed Jul. 11, 2006, and 60/859,772 filed Nov. 17, 2006, which are hereby incorporated by reference in their entirety.

### BACKGROUND

Controlled-release dosage formulations, including sustained-release formulations, provide a variety of benefits to the patient such as reduction in the number of doses per day, increased convenience, reduced occurrences of missed doses, and the chance to achieve controlled blood levels of the active agent.

Many dosage formulations result in the release of the active agent according to first-order kinetics. First-order release of the active agent results in initial high blood levels of the active agent followed by an exponential decrease in blood concentration. Peaks and spikes in blood levels of the active agent could result in an excess level above the therapeutic blood levels, perhaps even to toxic levels, followed by a rapid drop to blood levels below therapeutic levels.

Zero-order release kinetics result in blood levels of the active agent remaining constant through the time of delivery. Zero-order or near zero-order release has been achieved using osmotic devices. However, such osmotic devices are difficult to manufacture and are more expensive than traditional controlled-release technologies such as controlled-release coatings or controlled-release matrix formulations. Accordingly, there remains a need for other dosage formulations, in addition to osmotic forms, which provide zero-order or near zero-order release kinetics.

Fexofenadine ((±)-4-[1-hydroxy-4-[4-(hydroxydiphenylmethyl)-1-piperidinyl]-butyl]-α,α-dimethyl benzeneacetic acid) is a histamine H₁-receptor antagonist. It is often used in the treatment of histamine related illnesses.

Pseudoephedrine ([S—(R*,R*)]—α-[1-(methylamino)ethyl]-benzenemethanol) is an adrenergic (vasoconstrictor) agent, a sympathomimetic decongestant used for the relief of nasal congestion due to allergic rhinitis.

Although there exist combination forms of pseudoephedrine and fexofenadine, there is a need for improved pseudoephedrine and antihistamine dosage formulations, particularly dosage formulations having controlled-release of the pseudoephedrine with an immediate-release antihistamine portion. Also needed is a fexofenadine/pseudoephedrine dosage formulation having substantially no food effect such that a patient has the convenience of taking the dosage formulation with or without food.

### SUMMARY

In one embodiment, a controlled-release formulation comprises a core comprising a core active agent and a wax excipient; an extended-release coating substantially surrounding the core comprising a release-retarding coating material; and an immediate-release portion comprising an immediate-release active agent; wherein the release of the core active agent from the formulation is substantially zero-order, and wherein the core active agent and the immediate-release active agent are the same or different.

In another embodiment, a controlled-release formulation comprises a tablet core comprising pseudoephedrine or a pharmaceutically acceptable salt thereof, and a wax excipient; and an extended-release coating substantially surrounding the tablet core comprising a release-retarding coating material; and an immediate-release portion comprising an antihistamine as an immediate-release active agent; wherein the formulation exhibits substantially no food effect.

In yet another embodiment, a controlled-release formulation comprises a tablet core comprising pseudoephedrine or a pharmaceutically acceptable salt thereof, and a wax excipient; an extended-release coating substantially surrounding the tablet core comprising a release-retarding coating material; and an immediate-release portion comprising fexofenadine or a pharmaceutically acceptable salt thereof wherein the formulation is bioequivalent to a reference drug product according to New Drug Application No. 021704.

In still another embodiment, a controlled-release formulation comprises a tablet core comprising pseudoephedrine or a pharmaceutically acceptable salt thereof and a wax excipient; and an extended-release coating substantially surrounding the tablet core comprising a release-retarding coating material; wherein the formulation exhibits a dissolution profile such that at five hours after combining the formulation with 900 ml of purified water at 37° C.±0.5° C. according to USP 28 <711> test method 2 (paddle), 50 rpm paddle speed, about 10 to about 60 wt. % of the total amount of active agent is released. Dissolution is performed in 0.1; N HCl or pH 4.5 acetate buffer, for example.

In one embodiment, a controlled-release formulation comprises a core comprising a core active agent or a pharmaceutically acceptable salt thereof and a wax excipient; and an extended-release coating substantially surrounding the core comprising a release-retarding coating material; wherein the formulation exhibits a dissolution profile after combining the formulation with 900 ml of purified water at 37° C.±0.5° C. according to USP 28<711> test method 2 (paddle), 50 rpm paddle speed, wherein about 13 to about 40 wt. % of the total amount of the core active agent is released after three hours; about 20 to about 60 wt. % of the total amount of the core active agent is released after five hours; and about 40 to about 80 wt. % of the total amount of the core active agent is released after seven hours.

In another embodiment, a controlled-release formulation comprises a core comprising a core active agent or a pharmaceutically acceptable salt thereof and a wax excipient; and an extended-release coating substantially surrounding the core comprising a release-retarding coating material; wherein the formulation exhibits a dissolution profile after combining the formulation with 900 ml of a dissolution medium at 37° C.±0.5° C. according to USP 28<711> test method 2 (paddle), 50 rpm paddle speed, wherein about 15 to about 35 wt. % of the total amount of the core active agent is released after three hours; about 25 to about 50 wt. % of the total amount of the core active agent is released after five hours; about 35 to about 65 wt. % of the total amount of the core active agent is released after seven hours; and about 45 to about 75 wt. % of the total amount of the core active agent is released after nine hours.

In another embodiment, a controlled-release formulation comprises a tablet core comprising pseudoephedrine or a pharmaceutically acceptable salt thereof, and a wax excipient; an extended-release coating substantially surrounding the tablet core comprising a release-retarding coating material; and an immediate-release portion comprising fexofenadine or a pharmaceutically acceptable salt thereof wherein the formulation is bioequivalent to a reference drug product according to New Drug Application No. 020786.

These and other embodiments, advantages and features of the present invention become clear when detailed description and examples are provided in subsequent sections.

### BRIEF DESCRIPTION OF THE DRAWINGS

FIG. 1 illustrates a dissolution profile of extended-release coated pseudoephedrine cores and ALLEGRA-D® 24 HOUR.

FIG. 2 illustrates a dissolution profile of two additional extended-release coated pseudoephedrine cores.

### DETAILED DESCRIPTION

Disclosed herein are controlled-release formulations comprising a core comprising a core active agent and a wax excipient; and an extended-release coating substantially surrounding the core comprising a release-retarding coating material; wherein the release of the active agent from the formulation is substantially zero-order. It has been found that the particular combination of the wax matrix core and controlled-release coating provides a substantially linear release of the active agent from the core. Use of the wax matrix or the controlled-release coating alone does not provide a zero-order release.

Also disclosed herein are formulations comprising a core comprising a core active agent and a wax excipient; and an extended-release coating substantially surrounding the core comprising a release-retarding coating material; wherein the formulation exhibits substantially no food effect. By choosing the appropriate core materials and coating materials, the resulting formulation possesses enough strength to resist rupture or significant damage to the dosage formulation that could result in a compromise of the release properties. It has been found that a core of a particular hardness (“strength”, e.g., about 10 to about 15 kilopascals (kPa)) provides sufficient support for the controlled-release coating to ensure the integrity of the coating when the formulation is ingested with food.

In another embodiment, the formulation comprises a controlled-release portion in the form of a coated core and an immediate-release portion. The active agent present in the controlled-release portion and the immediate-release portion can be the same or different.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”). The endpoints of all ranges directed to the same component or property are inclusive and independently combinable.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

“Pharmaceutically acceptable salts” includes derivatives of the active agent, wherein the active agent is modified by making acid or base addition salts thereof, and further refers to pharmaceutically acceptable solvates, including hydrates, crystalline forms, non-crystalline forms, and polymorphs of such salts. Examples of pharmaceutically acceptable salts include, but are not limited to, mineral or organic acid addition salts of basic residues such as amines; alkali or organic addition salts of acidic residues; and the like, and a combination comprising at least one of the foregoing salts. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and a combination comprising at least one of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and a combination comprising at least one of the foregoing salts.

“Fexofenadine” means fexofenadine free base or a pharmaceutically acceptable fexofenadine salt, including any solvate, hydrate, crystalline form, and non-crystalline form thereof unless otherwise indicated.

A “dosage form” or “dosage formulation” means a unit of administration of an active agent. Examples of dosage formulations include tablets, capsules, injections, suspensions, liquids, emulsions, creams, ointments, suppositories, inhalable formulations, transdermal formulations, and the like. “Form” and “formulation” are to be used interchangeably unless indicated otherwise.

By “oral dosage form” is meant to include a unit dosage form for oral administration. An oral dosage form may optionally comprise a plurality of subunits such as, for example, microcapsules or microtablets. Multiple subunits may be packaged for administration in a single dose.

By “subunit” is meant to include a composition, mixture, particle, pellet, and the like, that can provide an oral dosage form alone or when combined with other subunits.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

“Food” typically means a solid food or mixed solid/liquid food with sufficient bulk and fat content that it is not rapidly dissolved and absorbed in the stomach. In one embodiment, food means a meal, such as breakfast, lunch or dinner. The terms “taken with food”, “fed” and “non-fasted” are equivalent and are as given by FDA guidelines and criteria. In one embodiment, with food means that the dosage form is administered to a patient between about 30 minutes prior to about 2 hours after eating a meal. In another embodiment, with food means that the dosage form is administered at substantially the same time as the eating the meal.

The terms “without food”, “fasted” and “an empty stomach” are equivalent and are as given by FDA guidelines and criteria. In one embodiment, fasted is means the condition wherein no food is consumed within 1 hour prior to administration of the dosage form or 2 hours after administration of the dosage form. In another embodiment, fasted means the condition wherein no food is consumed within 1 hour prior to administration of the dosage form to 2 hours after administration of the dosage form.

“Substantially no food effect” means that the pharmacokinetics are substantially the same for the oral administration of the formulation under fed conditions (“non-fasting”) when compared to administration under fasting conditions. For example, the comparison between Cₘₐₓ or AUC of a single administration of a formulation under fed conditions to a single administration of the same formulation under fasted conditions results in a percent ratio of Cₘₐₓ or AUC having a 90% confidence interval upper limit of less than or equal to 125% or a lower limit of greater than or equal to 80%. Such information can be based on logarithmic transformed data. Exemplary study considerations can be found in the Federal Drug Administration's (FDA) guidelines and criteria, including “Guidance for Industry, Food-Effect Bioavailability and Fed Bioequivalence Studies” available from the U.S. Department of Health and Human Services (DHHS), Food and Drug Administration (FDA), Center for Drug Evaluation and Research (CDER) December 2002, incorporated herein in its entirety.

A dissolution profile is a plot of the cumulative amount of active agent released from a formulation as a function of time. A dissolution profile can be measured utilizing the Drug Release Test <724>, which incorporates standard test USP 26 (Test <711>). A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

By “immediate-release” is meant a conventional or non-modified release in which greater then or equal to about 75% of the active agent is released within two hours of administration, specifically within one hour of administration.

By “controlled-release” is meant a dosage form in which the release of the active agent is controlled or modified over a period of time. Controlled can mean, for example, extended-, sustained-, delayed- or pulsed-release at a particular time. Alternatively, controlled can mean that the release of the active agent is extended for longer than it would be in an immediate-release dosage form, e.g., at least over several hours.

Dosage formulations can have both immediate-release and controlled-release characteristics, for example, a combination of immediate-release pellets and controlled-release pellets; immediate-release coating and controlled-release core including a tablet core; and the like. The immediate-release portion of a combination dosage form may be referred to as a loading dose.

ALLEGRA-D® 24 HOUR is an extended-release fexofenadine hydrochloride (180 mg)/pseudoephedrine hydrochloride (240 mg) oral tablet product marketed by Sanofi-Aventis and approved under the New Drug Application No. 021704.

ALLEGRA-D® 12 HOUR is an extended-release fexofenadine hydrochloride (60 mg)/pseudoephedrine hydrochloride (120 mg) oral tablet product marketed by Sanofi-Aventis and approved under the New Drug Application No. 020786.

Disclosed herein are fexofenadine and pseudoephedrine combination dosage formulations that exhibit substantially no food effect.

Also disclosed herein are fexofenadine and pseudoephedrine combination dosage formulations wherein the pseudoephedrine is released from the dosage form as substantially zero-order or near zero-order release, i.e., the release rate of the pseudoephedrine is substantially constant over time as determined by plotting the release of the active agent versus time. In specific embodiments, the release is zero-order over at least 8, 9, 10, 11, 12, 13, 14, 15, 16, 17, 18, 19, 20, 21, 22, 23 or 24 hours.

The core can be in the form of a particle, a pellet, a bead, a tablet, and the like, specifically as a tablet.

In some embodiments, the formulations described herein exhibit bioequivalence to the marketed drug product, for example ALLEGRA-D® 24 HOUR or ALLEGRA-D® 12 HOUR.

“Bioequivalence” means the absence of a significant difference in the rate and extent to which the active agent or surrogate marker for the active agent in pharmaceutical equivalents or pharmaceutical alternatives becomes available at the site of action when administered in an appropriately designed study.

In one embodiment, bioequivalence is any definition thereof as promulgated by the U.S. Food and Drug Administration or any successor agency thereof. In a specific embodiment, bioequivalence is determined according to the Federal Drug Administration's (FDA) guidelines and criteria, including “GUIDANCE FOR INDUSTRY BIOAVAILABILITY AND BIOEQUVALENCE STUDIES FOR ORALLY ADMINISTERED DRUG PRODUCTS-GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE” DHHS, FDA, CDER, January 2001, both of which are incorporated herein in their entirety.

In an embodiment, bioequivalence of a composition to a reference drug is determined by an in vivo pharmacokinetic study to determine a pharmacokinetic parameter for the active agent composition. Specifically, bioequivalence can be determined by an in vivo pharmacokinetic study comparing a pharmacokinetic parameter for the two compositions. A pharmacokinetic parameter for the active agent composition or the reference drug can be measured in a single or multiple dose bioequivalence study using a replicate or a nonreplicate design. For example, the pharmacokinetic parameters for active agent composition of the present invention and for a reference drug can be measured in a single dose pharmacokinetic study using a two-period, two-sequence crossover design. Alternately, a four-period, replicate design crossover study may also be used. Single doses of the test composition and reference drug are administered and blood or plasma levels of the active agent are measured over time. Pharmacokinetic parameters characterizing rate and extent of active agent absorption are evaluated statistically.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA).

Under U.S. FDA guidelines, two products (e.g., an inventive fexofenadine formulation and ALLEGRA-D® 24 Hour) or methods (e.g., dosing under non-fasted versus fasted conditions) are bioequivalent if the 90% Confidence Interval (CI) limits for a ratio of the geometric mean of logarithmic transformed AUC_{0-∞}, AUC₀₋ₜ, and Cₘₐₓ for the two products or two methods are about 0.80 to about 1.25.

In another embodiment, bioequivalence is determined according to the European Medicines Agency (EMEA) document “Note for Guidance on the Investigation of Bioavailability and Bioequivalence”, issued Jul. 26, 2001, available from EMEA.

To show bioequivalency between two compounds or administration conditions pursuant to Europe's EMEA guidelines, the 90% CI limits for a ratio of the geometric mean of logarithmic transformed AUC_{0-∞} and AUC₀₋ₜ for the two products or methods are about 0.80 to about 1.25. The 90% CI limits for a ratio of the geometric mean of logarithmic transformed Cₘₐₓ for the two products or methods can have a wider acceptance range when justified by safety and efficacy considerations. For example the acceptance range can be about 0.70 to about 1.43, specifically about 0.75 to about 1.33, and more specifically about 0.80 to about 1.25.

In one embodiment, in a given experiment, an active agent composition is considered to be bioequivalent to the reference product if both the Test/Reference ratio for the geometric mean of logarithmic transformed AUC_{0-∞}, AUC₀₋ₜ, or Cₘₐₓ ratio along with its corresponding lower and upper 90% CI limits are within a lower limit of about 0.80 and an upper limit of about 1.25. Thus, for direct comparison between an inventive active agent composition and a reference product, the pharmacokinetic parameters for the active agent composition and the reference product can be determined in side-by side in the same pharmacokinetic study.

In some embodiments a single dose bioequivalence study is performed under non-fasted or fasted conditions.

In other embodiments, the single dose bioequivalence study is conducted between the active agent composition and the reference listed drug using the strength specified by the FDA in APPROVED DRUG PRODUCTS WITH THERAPEUTIC EQUIVALENCE EVALUATIONS(ORANGE BOOK).

In some embodiments, an in vivo bioequivalence study is performed to compare all active agent compositions with corresponding strengths of the reference product (e.g., 60, 120, or 180 mg of the active agent). In other embodiments, an in vivo bioequivalence study is performed only for the active agent composition of the present invention at the strength of the reference listed drug product (e.g., the highest approved strength) and at the other lower or higher strengths, the inventive compositions meet a reference product dissolution test.

In one embodiment, a controlled-release formulation comprises fexofenadine and pseudoephedrine, wherein the formulation exhibits a ratio of a geometric mean of logarithmic transformed AUC_{0-∞} of the controlled-release formulation to a geometric mean of logarithmic transformed AUC_{0-∞} of fexofenadine/pseudoephedrine reference drug approved under the New Drug Application No. 021704 of about 0.80 to about 1.25.

In another embodiment, a controlled-release formulation comprises fexofenadine and pseudoephedrine, wherein the formulation exhibits a ratio of a geometric mean of logarithmic transformed AUC_{0-∞}T of the controlled-release formulation to a geometric mean of logarithmic transformed AUC₀₋ₜ of fexofenadine/pseudoephedrine reference drug approved under the New Drug Application No. 021704 of about 0.80 to about 1.25.

In yet another embodiment, a controlled-release formulation comprises fexofenadine, wherein the formulation exhibits a ratio of a geometric mean of logarithmic transformed Cₘₐₓ of the controlled-release formulation to a geometric mean of logarithmic transformed Cₘₐₓ of fexofenadine/pseudoephedrine reference drug approved under the New Drug Application No. 021704 of about 0.70 to about 1.43.

In yet another embodiment, a controlled-release formulation comprises fexofenadine and pseudoephedrine, wherein the formulation exhibits a ratio of a geometric mean of logarithmic transformed Cₘₐₓ of the controlled-release formulation to a geometric mean of logarithmic transformed Cₘₐₓ of fexofenadine/pseudoephedrine reference drug approved under the New Drug Application No. 021704 of about 0.80 to about 1.25.

Suitable active agents for the core or immediate-release coating can include, for example, an alpha-2 adrenergic agent, an analgesic, an angiotensin-converting enzyme (ACE) inhibitor, an antianxiety agent, an antiarrhythmic, an antibacterial, an antibiotic, an antidepressant, antidiabetics, an antiemetic, an antiepileptic, an antifungal, an antihelminthic, an antihistamine, an antihyperlipidemic, an antihypertensive, an antiinfective, an antimalarial, an antimicrobial, an antimigraine agent, an antimuscarinic agent, an antineoplastic, an antiprotozoal, an antipsychotic, an antispasmodic, an antiviral, an attention-deficit hyperactivity disorder (ADHD) agent, a 0-blocker, a calcium channel blocker, achemotherapeutic agent, a cholinesterase inhibitor, a Cox-2 inhibitor, a decongestant, a diuretic, a histamine-2 receptor antagonist, a hypnotic, a hypotensive agent, an immunosuppressant, a lipotropic, a neuroleptic, an opioid analgesic, a peripheral vasodilator/vasoconstrictor, a sedative, a serotonin receptor agonist, a sympathomimetic, a pharmaceutically acceptable salt, solvate, hydrate, stereoisomer (racemate, individual enantiomer or diastereomer, or any combination thereof), or polymorph thereof, or a pharmaceutically acceptable combination comprising at least one of the foregoing active agents, and the like.

Exemplary pharmaceutically active agents include amphetamine, brompheniramine, cetirizine, chlorpheniramine, clemastine, desloratadine, dextroamphetamine, diltiazem, diphenhydramine, fexofenadine, fluvastatin, guaifenesin, hydromorphone, loratidine, morphine, oxybutynin, oxycodone, paroxetine, propranolol, pseudoephedrine, terphenadine, tolterodine, venlafaxine, sulfamethoxazole, trimethoprim, a pharmaceutically acceptable salt, solvate, hydrate, stereoisomer (racemate, individual enantiomer or diastereomer, or any combination thereof), or polymorph thereof, or a pharmaceutically acceptable combination comprising at least one of the foregoing active agents.

Specifically, the active agent is pseudoephedrine ([S—(R*,R*)]-α-[1-(methylamino)ethyl]-benzenemethanol), a pseudoephedrine salt (e.g., hydrochloride), fexofenadine ((±)-4-[1-hydroxy-4-[4-(hydroxydiphenylmethyl)-1-piperidinyl]-butyl]-α,α-dimethyl benzeneacetic acid), a fexofenadine salt (e.g., hydrochloride), and combinations thereof. Fexofenadine, including fexofenadine hydrochloride, is known to exist in many different polymorphic forms and it has been surprisingly shown by the inventors herein that the particular polymorphic form of fexofenadine can have significant effects on the properties of a fexofenadine dosage form, such as, for example, dissolution. Suitable fexofenadine free base and salts include those disclosed in U.S. patent and patent application Publication Nos. 6,613,906, 2002193603 (anhydrous fexofenadine hydrochloride Form I and Form III and hydrated fexofenadine hydrochloride Form II and Form IV), US2002193601, US2005090528 (fexofenadine free base), US20020177608, US2003021849, US20040044038, US2004058955, US2004167168, US2004077683, US2004248935, US2005090528, US20050165056, US20050256163, US20050282860, and US20060025444, as well as PCT publications WO2005019175 and WO2006037042; each of which is incorporated herein by reference in its entirety.

The formulations disclosed herein comprise a core comprising an active agent, a wax excipient, and optionally additional core excipients.

The wax excipient for use in the core can be a solid wax at ambient temperature, such as a solid, hydrophobic material (i.e., non-water soluble) or solid hydrophilic material (e.g., polyethylene glycols are water soluble), but specifically a solid, hydrophobic material.

Exemplary wax excipients include wax and wax-like excipients, for example, carnauba wax (from the palm tree Copernicia Cerifera), vegetable wax, fruit wax, microcrystalline wax (“petroleum wax”), bees wax (white or bleached, and yellow), hydrocarbon wax, paraffin wax, cetyl esters wax, non-ionic emulsifying wax, anionic emulsifying wax, candelilla wax, or a combination comprising at least one of the foregoing waxes. Other suitable wax excipients include, for example, fatty alcohols (such as lauryl, myristyl, stearyl, cetyl or specifically cetostearyl alcohol), hydrogenated vegetable oil, hydrogenated castor oil, fatty acids such as stearic acid, fatty acid esters including fatty acid glycerides (mono-, di-, and tri-glycerides), polyethylene glycol (PEG) having a molecular weight of greater than about 3000 number average molecular weight, Mₙ (e.g., PEG 3350, PEG 4000, PEG 4600, PEG 6000, and PEG 8000), or a combination comprising at least one of the foregoing wax excipients. Any combination of wax excipients is also contemplated.

The melting point of the wax excipient is a temperature above ambient temperature, specifically about 30 to about 150° C., more specifically about 75 to about 100° C., and yet more specifically about 75 to about 90° C.

The amount of wax excipient present in the core can be determined based on the particular wax or wax combination chosen and the targeted release profile desired for the resulting formulation. Exemplary amounts of a wax excipient include about 5 to about 60 wt. % based on the total weight of the core excluding the extended-release coating, specifically about 10 to about 50 wt. %, and more specifically about 15 to about 40 wt. %.

The core also comprises a core active agent such as pseudoephedrine. Exemplary amounts of active agent in the core include about 30 to about 60 wt. % based on the total weight of the core excluding the extended-release coating, specifically about 35 to about 50 wt. %, and more specifically about 40 to about 45 wt. %.

The core optionally further contains an additional release-retarding material. Additional release-retarding materials include, for example an acrylic polymer, an alkylcellulose including substituted alkylcellulose, shellac, zein, polyvinylpyrrolidine including crosslinked polyvinylpyrrolidinone, a vinyl acetate copolymer, a polyethylene oxide, a polyvinyl alcohol, and a combination comprising at least one of the foregoing materials.

Suitable acrylic polymers for use as an additional release-retarding material include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, or a combination comprising at least one of the foregoing polymers. The acrylic polymer may comprise methacrylate copolymers described in NF XXIV as fully polymerized copolymers of acrylic and methacrylic acid esters with a low content of quaternary ammonium groups.

Suitable alkylcelluloses and substituted alkyl celluloses include, for example, methyl cellulose, ethylcellulose, hydroxy or carboxy substituted alkyl celluloses (e.g., hydroxylpropylcellulose, crosslinked hydroxypropylcellulose, carboxymethylcellulose, crosslinked sodium carboxymethylcellulose), hydroxy substituted alkyl-alkyl celluloses (e.g., hydroxypropylmethylcellulose), or a combination comprising at least one of the foregoing alkyl celluloses.

The additional release-retarding material is present in the core in an amount of 0 to about 65 wt. % based on the total weight of the core, specifically about 0.1 to about 50 wt. %, more specifically about 10 to about 45 wt. %, and yet more specifically about 15 to about 30 wt. %. Besides the additional release-retarding material, the additional core excipients optionally includes binders, fillers, disintegrants, lubricants, glidants, and the like.

The optional disintegrant is used to facilitate the breakdown of the core in a fluid environment, specifically aqueous environments. The choice and amount of disintegrant is tailored to ensure the desired dissolution profile of the formulation or to provide the desired controlled-release in vivo. Exemplary disintegrants include a material that possesses the ability to swell or expand upon exposure to a fluid environment, especially an aqueous environment. Exemplary disintegrants include hydroxyl substituted alkyl celluloses (e.g., hydroxypropyl cellulose), starch, pregelatinized starch (e.g., Starch 1500® available from Colorcon); cross-linked sodium carboxymethylcellulose (e.g., “croscarmellose sodium”, i.e., Ac-Di-Sol® available from FMC BioPolymer of Philadelphia, Pa.); crosslinked homopolymer of N-vinyl-2-pyrrolidone (e.g., “crospovidone”, e.g., Polyplasdone® XL, Polyplasdone® XL-10, and Polyplasdone® INF-10 available from International Specialty Products, Wayne N.J.); modified starches, such as sodium carboxymethyl starch, sodium starch glycolate (e.g., Primogel®), and the like; alginates; or a combination comprising at least one of the foregoing disintegrants.

The amount of disintegrant used depends upon the disintegrant or disintegrant combination chosen and the targeted release profile of the resulting formulation. Exemplary amounts include about 0 to about 10 wt. % based on the total weight of the core, specifically about 0.5 to about 7.0 wt. %, and yet more specifically about 0.1 to about 5.0 wt. %.

Exemplary lubricants include stearic acid, stearates (e.g., calcium stearate, magnesium stearate, and zinc stearate), sodium stearyl fumarate, glycerol behenate, mineral oil, polyethylene glycols, talc, hydrogenated vegetable oil, vegetable based fatty acids, or a combination comprising at least one of the foregoing. Glidants include, for example, silicon dioxide (e.g., fumed or colloidal). It is recognized that certain materials can function both as a glidant and a lubricant.

The lubricant or glidant is used in amounts of about 0.1 to about 15 wt. % of the total weight of the core; specifically about 0.5 to about 5 wt. %; and yet more specifically about 0.75 to about 3 wt. %.

The cores are prepared by processes known in the art, including granulation (dry or wet) and compression, spheronization, melt extrusion, hot fusion, and the like.

Once the core is formed, it is coated with an extended-release coating. The extended-release coating that substantially surrounds the core comprises a release-retarding coating material and optional other components, such as plasticizers, pore formers, and the like.

The extended-release coating is present in the formulation at about 0.1 to about 30 wt. % based on the total weight of the core and extended-release coating, specifically about 3.0 to about 25 wt. %, more specifically about 4.0 to about 20 wt. %, and yet more specifically about 5.0 to about 20 wt. %.

The extended-release coating is provided on the core using known coating processes such as simple or complex coacervation, interfacial polymerization, liquid drying, thermal and ionic gelation, spray drying, spray chilling, fluidized bed coating, pan coating, electrostatic deposition, compression coating, dry polymer powder coating, and the like.

The release-retarding coating material is, for example, in the form of a film coating comprising a dispersion of a hydrophobic polymer. Solvents used for application of the controlled-release coating include pharmaceutically acceptable solvents, such as water, methanol, ethanol, methylene chloride, and a combination comprising at least one of the foregoing solvents.

The extended-release profile of the active agent (either in vivo or in vitro) can be altered, for example, by using more than one release-retarding coating material, varying the thickness of the release-retarding coating material, changing the particular release-retarding coating material used, altering the relative amounts of release-retarding coating material, use of a plasticizer, altering the manner in which the plasticizer is added (e.g., when the extended-release coating is derived from an aqueous dispersion of hydrophobic polymer), by varying the amount of plasticizer relative to release-retarding coating material, by the inclusion of an additional coating excipient, by altering the method of manufacture, and the like.

Exemplary release-retarding coating materials include film-forming polymers such as an alkylcellulose including methylcellulose or ethylcellulose, a hydroxyalkylcellulose such as hydroxymethyl cellulose, hydroxyethylcellulose, hydroxypropylcellulose and hydroxybutylcellulose, a hydroxyalkyl alkylcellulose such as hydroxyethyl methylcellulose and hydroxypropyl methylcellulose, a carboxyalkylcellulose such as carboxymethylcellulose, an alkali metal salt of carboxyalkylcelluloses such as sodium carboxymethylcellulose, a carboxyalkyl alkylcellulose such as carboxymethyl ethylcellulose, a carboxyalkylcellulose ester, a starch, a pectin such as sodium carboxymethylamylopectine, a chitin derivate such as chitosan, a polysaccharide such as alginic acid, alkali metal and ammonium salts thereof, a carrageenan, a galactomannan, traganth, agar-agar, gum arabicum, guar gum and xanthan gum, a polyacrylic acid and the salts thereof, a polymethacrylic acid and the salts thereof, a methacrylate copolymer, a polyvinylalcohol, a polyvinylpyrrolidone, a copolymer of polyvinylpyrrolidone with vinyl acetate, a polyalkylene oxide such as polyethylene oxide and polypropylene oxide and a copolymer of ethylene oxide and propylene oxide, or a combination comprising at least one of the foregoing materials.

The controlled-release coating optionally comprises a plasticizer, an additional film-former, a pore former, or a combination comprising at least one of the foregoing materials.

The formulations optionally further comprises a non-functional coating. By “functional coating” is meant to include a coating that modifies the release properties of the total formulation, for example, a controlled-release coating that provides sustained-release. By “non-functional coating” is meant to include a coating that does not significantly modify the release properties of the total formulation, for example, a cosmetic coating or an interlayer coating used to separate a functional coating from other components of the formulation. A non-functional coating can have some impact on the release of the active agent due to the initial dissolution, hydration, perforation of the coating, and the like, but would not be considered to be a significant deviation from the non-coated composition.

A pore forming material is optionally be added to the controlled-release coating to promote release of the active agent from the core. The pore forming material is organic or inorganic; it is a material that can be dissolved, extracted or leached from the coating in the environment of use; or it can have a pH-dependent solubility property; and the like. Exemplary pore forming materials include hydrophilic polymers such as a hydroxy alkyl-alkyl cellulose (e.g., hydroxypropylmethyl cellulose, and the like), a hydroxyl alkyl cellulose (e.g., hydroxypropylcellulose, and the like), or a povidone; a saccharide (e.g., lactose, and the like); a metal stearate; an inorganic salt (e.g., dibasic calcium phosphate, sodium chloride, and the like); a polyethylene glycol (e.g., polyethylene glycol (PEG) 1450, and the like); a sugar alcohol (e.g., sorbitol, mannitol, and the like); an alkali alkyl sulfate (e.g., sodium lauryl sulfate); a polyoxyethylene sorbitan fatty acid ester (e.g., polysorbate); methyacrylate copolymers (e.g., EUDRAGIT® RL); or a combination comprising at least one of the foregoing pore forming materials.

A specific release-retarding coating material includes ethyl cellulose and optionally in combination with hydroxypropylmethyl cellulose.

In one embodiment, the ratio of ethyl cellulose to hydroxypropylmethyl cellulose is about 90:10, specifically about 60:40, and yet more specifically about 50:50.

In one embodiment, the controlled-release core releases the core active agent over a period of about 6 hours to about 24 hours, specifically about 12 hours or about 24 hours.

In one embodiment, the formulation comprises a controlled-release portion in the form of a coated core and an immediate-release portion disposed on at least a portion of the controlled-release core. The formulation is prepared, for example, as a bilayer tablet, a coated tablet, a compression-coated tablet, or any other suitable form. In one embodiment, the immediate-release portion is in the form of a coating substantially surrounding the coated core applied, for example, using spray coating, compression coating, or other suitable technique. The active agent present in the controlled-release portion and the immediate-release portion can be the same or different. In one embodiment, the active agent in the controlled-release portion is pseudoephedrine and the active agent in the immediate-release portion is fexofenadine. The pseudoephedrine is present in the formulation in an amount of 80 to 300 mg, specifically 60-240 mg, more specifically 120 or 240 mg. The fexofenadine is present in the formulation in an amount of 40 to 250 mg, specifically 60 to 180 mg, more specifically 60 or 180 mg.

In one embodiment, the immediate-release portion of the formulation is in the form of a coating comprising an active agent such as fexofenadine and one or more excipients such as a disintegrant (e.g., crospovidone, croscarmellose sodium, pregelatinized starch), a binder (e.g., povidone), a lubricant (e.g., magnesium stearate, polyethylene glycol), a filler (e.g., microcrystalline cellulose, colloidal silicon dioxide), solubilizing and/or wetting agents (e.g., polysorbate 80), and combinations comprising one or more of the foregoing excipients.

It has been unexpectedly discovered by the inventors herein that the release properties of fexofenadine hydrochloride from an immediate-release composition are affected by the hydration state and/or polymorphic form of the fexofenadine hydrochloride. In one embodiment, the fexofenadine hydrochloride comprises substantially no anhydrous fexofenadine hydrochloride Form I. By substantially no anhydrous fexofenadine hydrochloride Form I it is meant that the fexofenadine hydrochloride is missing at least one of the following major x-ray diffraction peaks: 11.8, 7.3, 6.3, 5.9, 5.0, 4.8, 4.4, 3.9, 3.8 and 3.7 Angstroms. In another embodiment, fexofenadine hydrochloride comprises substantially no anhydrous fexofenadine hydrochloride Form I or Form II. By substantially no anhydrous fexofenadine hydrochloride Form II it is meant that the fexofenadine hydrochloride is missing at least one of the following major x-ray diffraction peaks: 7.8, 6.4, 5.2, 4.9, 4.7, 4.4, 4.2, 4.1, 3.7, 3.6, and 3.5 Angstroms.

In one embodiment, the fexofenadine hydrochloride is substantially Form C fexofenadine hydrochloride as described in WO2005019175, incorporated herein by reference in its entirety. By substantially Form C fexofenadine hydrochloride it is meant that the fexofenadine hydrochloride has characteristic peaks at: 9.85, 5.97, 5.52, 5.19, 4.83, 4.59, 4.49, 4.20, 4.13, 3.85 and 3.73 Angstroms; or 8.9712, 14.8293, 16.0514, 17.0775, 18.3418, 19.3099, 19.7703, 21.1340, 21.5207, 23.0743 and 23.8286 degrees two theta.

In one embodiment, the fexofenadine hydrochloride is substantially Form X fexofenadine hydrochloride as described in US 20040077683 and US20040058955, incorporated herein by reference in its entirety. By substantially Form X fexofenadine hydrochloride it is meant that the fexofenadine hydrochloride has characteristic peaks at: 16.05, 12.98, 8.29, 8.06, 6.25, 5.97, 5.54, 5.41, 4.89, 4.70, 4.55, 4.37, 4.32, 4.15, 4.03, 3.80, 3.67, 3.57 and 3.42 Angstroms; or 4.2, 8.0, 9.3, 14.2, 16.0, 16.8, 17.2, 17.6, 18.8, 20.0, 20.6, 21.7, 22.9, 23.8, 24.2, and 24.4 degrees two theta.

In another embodiment, the fexofenadine and one or more excipients are deposited (e.g., sprayed) onto the tablet cores to form a substantially continuous coating. In a specific embodiment, the fexofenadine is an anhydrous form of fexofenadine hydrochloride and the anhydrous fexofenadine hydrochloride and the one or more excipients is deposited onto the cores in the form of a solution or suspension comprising a nonaqueous solvent such as, for example, isopropyl alcohol.

In one embodiment, the polymorphic form of the fexofenadine hydrochloride prior to deposition on the core is substantially the same as the polymorphic form of the fexofenadine hydrochloride after deposition on the core. That is, the deposition process does not substantially alter the polymorphic form of the fexofenadine hydrochloride. By substantially the same, it is meant that less than 5%, less than 2%, less than 1%, or less than 0.5% of the fexofenadine changes its polymorphic form upon deposition.

The pseudoephedrine/fexofenadine formulations disclosed herein can exhibit an in vitro dissolution profile substantially similar to the dissolution profile achieved by the ALLEGRA-D® 24 HOUR product NDA No. 021704 when tested in a desired dissolution medium.

Also included herein are pharmaceutical kits which comprise one or more containers containing a controlled-release formulation, wherein the formulation comprises a core comprising an active agent and a wax excipient; and an extended-release coating substantially surrounding the core comprising a release-retarding coating material. The kits may further comprise one or more conventional pharmaceutical kit components, such as, for example, one or more containers to aid in facilitating compliance with a particular dosage regimen; one or more carriers; printed instructions, either as inserts or as labels, indicating quantities of the components to be administered, or guidelines for administration. Exemplary kits can be in the form of bubble or blister pack cards, optionally arranged in a desired order for a particular dosing regimen. Suitable blister packs that can be arranged in a variety of configurations to accommodate a particular dosing regimen are well known in the art or easily ascertained by one of ordinary skill in the art.

### EXAMPLES

### Example 1

### Preparation of Pseudoephedrine HCl Extended-Release Tablet Cores, 240 mg

Extended-release pseudoephedrine HCl tablet cores (drug core and extended-release coating) are prepared having the components listed in Table 1 below.

TABLE 1

| Component | milligram/tablet | | | |
|---|---|---|---|---|
| Core Formulation | A | B | C | D |
| Pseudoephedrine HCl | 240 | 240 | 240 | 240 |
| Carnauba Wax, NF | 134 | 134 | 134 | 134 |
| Stearic Acid, NF | 90 | 90 | 90 | 90 |
| Denatured Alcohol | * | * | * | * |
| Hydroxy Propylcellulose, NF | 91 | 91 | 91 | 91 |
| (Klucel ® HXF) | | | | |
| Silicon Dioxide, NF | 5 | 5 | 5 | 5 |
| (Syloid ® 244 FP) | | | | |
| Magnesium Stearate, NF | 5 | 5 | 5 | 5 |
| Core coating | | | | |
| Surelease ® (25% w/w | 51.35 | 23.4 | 39.5 | 33.9 |
| ethylcellulose) | | | | |
| Opadry ® Clear # YS-3-7011 | 27.65 | 15.6 | 39.5 | 22.6 |
| Purified Water, USP | * | * | * | * |

* Not found in final product

The tablet cores are prepared by granulating pseudoephedrine HCl and carnauba wax in a mixer/granulator for five minutes. Stearic acid is dissolved in denatured alcohol with mixing and gentle heat. The stearic acid mixture is added to the active agent/wax mixture and mixed to form granules. The resulting granules are dried and milled. The milled granules are charged to a Gemco Blender to which screened hydroxylpropylcellulose and silicon dioxide are added and mixed. Screened magnesium stearate is then added and mixed to form a blend. The resulting blend is then compressed into tablets. The compressed tablets are coated with a core coating prepared from a suspension of Surelease®, Opadry® Clear and water to form extended-release pseudoephedrine tablet cores.

The extended-release tablet cores of Formulation A-D of Table 1 were analyzed for in vitro dissolution using the test method protocol according to USP 26, 711 using 900 milliliters of a dissolution medium at 37° C.±0.5° C. and a paddle speed of 50 rotations per minute (rpm). The results of the dissolution analyses are summarized in Tables 2a and 2b below, including results from testing uncoated cores from Example 1 as well as samples of ALLEGRA-D® 24 HOUR.

TABLE 2a

| | | % Pseudoephedrine released, purified water | | | |
|---|---|---|---|---|---|
| | | | Formulation | Formulation | Formulation |
| Time | ALLEGRA-D ® | Uncoated Cores, | A (14% | B (7% | C (14% |
| (hr) | 24 HOUR | Example 1 | coating) | coating) | coating) |
| 0 | 0 | 0 | 0 | 0 | 0 |
| 1 | 2 | 35 | 3 | 13 | 16 |
| 2 | 10 | 51 | 8 | 20 | 30 |
| 3 | 19 | 63 | 13 | 28 | 40 |
| 4 | 25 | 72 | 19 | — | — |
| 5 | 33 | 80 | 24 | 40 | 58 |
| 6 | 40 | 86 | 28 | — | — |
| 7 | — | — | — | 53 | 72 |
| 8 | 55 | 95 | 36 | — | — |
| 9 | — | — | — | 63 | 83 |
| 11 | — | — | — | 73 | 91 |
| 12 | 82 | 103 | 52 | — | — |
| 13 | | | | 81 | 96 |
| 14 | 86 | 105 | 60 | — | — |
| 15 | — | — | — | 88 | 100 |
| 23 | — | — | — | 103 | 107 |
| 24 | 96 | 108 | 87 | — | — |

TABLE 2b

| | | % Pseudoephedrine released; Formulation D (10% coating) | | | |
|---|---|---|---|---|---|
| | ALLEGRA-D ® | | | Acetate | Phosphate |
| Time | 24 HOUR | | | buffer | buffer |
| (hr) | (Water) | Water | 0.1N HCl | (pH 4.5) | (pH 6.8) |
| 0 | 0 | 0 | 0 | 0 | 0 |
| 1 | 7 | 10 | 9 | 11 | 10 |
| 2 | 15 | 17 | 17 | 19 | 20 |
| 3 | 22 | 24 | 24 | 26 | 28 |
| 5 | 38 | 36 | 38 | 38 | 43 |
| 7 | 51 | 45 | 52 | 49 | 55 |
| 9 | 64 | 53 | 64 | 59 | 65 |
| 11 | — | — | 74 | 69 | 72 |
| 13 | — | — | 82 | 77 | 78 |
| 15 | — | — | 89 | 84 | 84 |
| 23 | 96 | 95 | 105 | 100 | 95 |

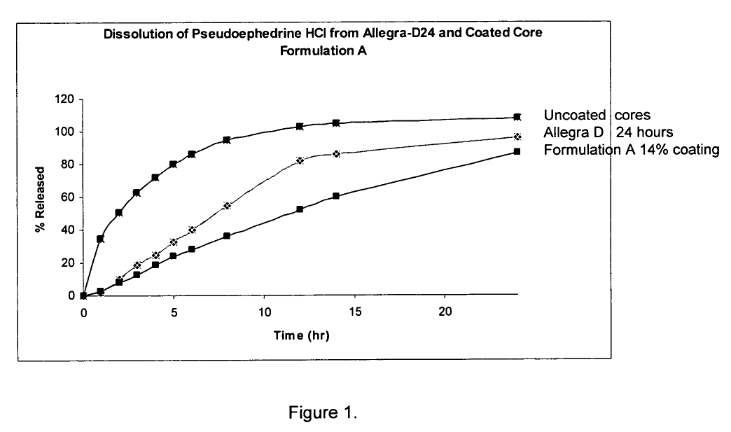

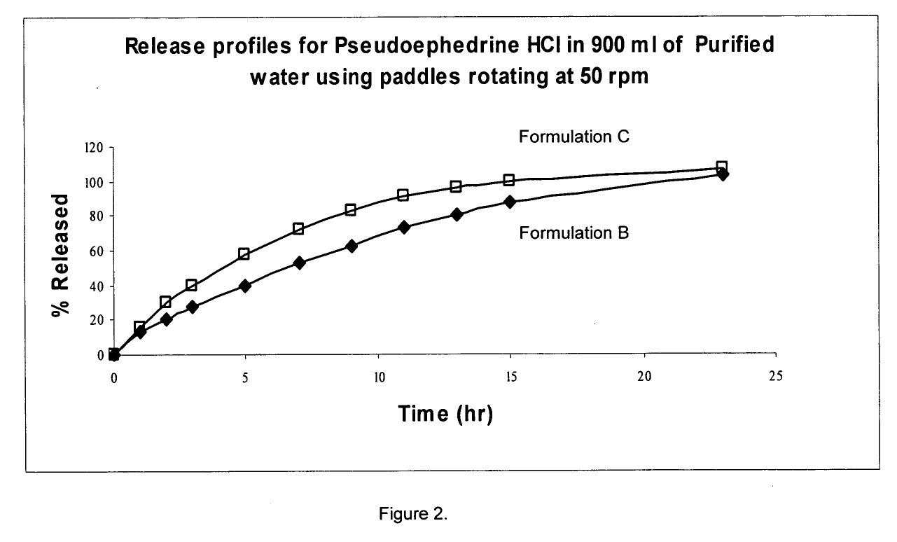

As the dissolution results in Table 2a and FIGS. 1-2 indicate, the extended-release pseudoephedrine tablet cores of Formulations A-C exhibit substantially zero-order or near zero-order release profiles.

Table 2b contains dissolution results for Formulation D in a variety of dissolution media. As indicated by the results, the release of the active agent is substantially independent of the pH of the media and is substantially zero-order.

### Example 2

### Fexofenadine HCl/Pseudoephedrine HCl Extended Release Tablets, 180 mg/240 mg

An extended-release dosage form is prepared having an immediate-release fexofenadine HCl coating over an extended-release pseudoephedrine HCl tablet core.

A spray coat formulation of fexofenadine HCl is prepared having the components of Table 3 below:

TABLE 3

| Fexofenadine coating | milligram/tablet |
|---|---|
| Fexofenadine HCl | 180 |
| Plasdone ® S-630, NF (Povidone S-630) | 62.5 |
| Polyplasdone ® XL10, NF (Crospovidone) | 57.5 |
| Carbowax ™ 400, NF (Polyethylene Glycol 400) | 6 |
| Polysorbate 80, NF (Tween ® 80) | 7 |
| Colloidal Silicon Dioxide, NF (Cab-O-Sil ®) | 7 |
| Isopropyl Alcohol | * |

* Not found in final product

The pseudoephedrine tablet cores of Example 1 are spray coated with the fexofenadine coating formulation according to Table 3 to result in an amount sufficient to result in about 180 mg of fexofenadine per tablet. Suitable spray coating techniques known to one of ordinary skill in the art can be used.

### Example 3

### Fexofenadine HCl/Pseudoephedrine HCl Extended Release Tablets, 180 mg/240 mg

An extended-release dosage form is prepared having an immediate-release fexofenadine HCl compression coating over an extended-release pseudoephedrine HCl tablet core.

A compression coat formulation of fexofenadine HCl is prepared having the components of Table 4 below:

TABLE 4

| Fexofenadine coating | milligram/tablet |
|---|---|
| Fexofenadine HCl | 180 |
| Microcrystalline cellulose, NF (Avicel ® pH 101) | 127.5 |
| Pregelatinized Starch, NF (Starch 1500 ®) | 40 |
| Croscarmellose Sodium, NF (Ac-Di-Sol ®) | 30 |
| Magnesium stearate | 2.5 |

The pseudoephedrine core tablets of Example 1 are compression coated with the fexofenadine coating formulation according to Table 4 to result in an amount sufficient to result in about 180 mg of fexofenadine per tablet.

Optionally, instead of the dry blend as used in Table 4, the compression coating can include water, isopropyl alcohol (anhydrous), ethanol (anhydrous), or other pharmaceutically acceptable liquid to form a wet blend that is granulated prior to compression coating for ease of formulating. Suitable compression coating techniques known to one of ordinary skill in the art can be used. The granulating liquid is not present in the final dosage form.

### Example 4

### Fexofenadine HCl/Pseudoephedrine HCl Extended Release Tablets, 180 mg/240 mg

An extended-release dosage form (Formulation N) is prepared having an immediate-release fexofenadine HCl coating over an extended-release pseudoephedrine HCl tablet core. The pseudoephedrine core is formed as in Example I and the fexofenadine coating is deposited by spray drying in a pan.

TABLE 5
Pseudoephedrine core

| Core Formulation | Mg/tablet |
|---|---|
| Pseudoephedrine HCl | 240 |
| Carnauba Wax, NF | 134 |
| Stearic Acid, NF | 90 |
| Denatured Alcohol | * |
| Hydroxy Propylcellulose, NF (Klucel ® HXF) | 91 |
| Silicon Dioxide, NF (Syloid ® 244 FP) | 5 |
| Magnesium Stearate, NF | 5 |
| Core coating | |
| Surelease ® (25% w/w ethylcellulose) | 200 |
| Opadry ® Clear # YS-3-7011 | 50 |
| Purified Water, USP | * |

TABLE 6
Fexofenadine coating

| Fexofenadine coating | milligram/tablet |
|---|---|
| Fexofenadine HCl | 180 |
| Hypromellose (Methocel E3 Premium LV) | 5 |
| Carbowax ™ 400, NF (Polyethylene Glycol 400) | 10 |
| Croscarmelose Sodium (Ac-Di-Sol ®) | 5 |
| Purified water | 12 |
| Isopropyl Alcohol | * |

* Not found in final product

The tablets comprising pseudoephedrine and fexofenadine are then overcoated with a nonfunctional coating such as about 3 wt. % Opadry® clear or about 3 wt. % Opadry® white.

The release of fexofenadine from this exemplary formulation was measured according to USP 26, 711 using 900 milliliters of a dissolution medium at 37° C.±0.5° C. and a paddle speed of 50 rotations per minute (rpm) in 0.1 N HCl or pH 4.5 acetate buffer. The results are given in Tables 11 and 12 and compared to ALLEGRA D® 24 hour.

TABLE 7
0.1 N HCl

| | | | Formulation | Formulation |
|---|---|---|---|---|
| | | | N with | N with |
| | ALLEGRA | Formulation | Opadry ® | Opadry ® |
| Time, min | D ® 24 HR | N | Clear | White |
| 0 | 0 | 0 | 0 | 0 |
| 5 | 24 | 77 | 2 | 25 |
| 10 | 68 | 99 | 53 | 78 |
| 15 | 96 | 105 | 76 | 93 |
| 30 | 102 | 107 | 86 | 100 |
| 45 | 101 | 107 | 91 | 104 |
| 60 | 101 | 108 | 93 | 106 |
| 120 | 100 | 107 | 98 | 110 |

TABLE 8
pH 4.5 acetate buffer

| | | | Formulation | Formulation |
|---|---|---|---|---|
| | | | N with | N with |
| | ALLEGRA | Formulation | Opadry ® | Opadry ® |
| Time, min | D ® 24 HR | N | Clear | White |
| 0 | 0 | 0 | 0 | 0 |
| 5 | 5 | 85 | 0 | 3 |
| 10 | 24 | 97 | 24 | 33 |
| 15 | 47 | 99 | 54 | 55 |
| 30 | 75 | 102 | 71 | 65 |
| 45 | 80 | 102 | 77 | 69 |
| 60 | 82 | 102 | 80 | 70 |
| 120 | 85 | 103 | 84 | 74 |

As can be seen in Tables 7 and 8, adding a nonfunctional overcoat over the fexofenadine coating does not substantially alter the release of the fexofenadine from the dosage form after the first 10-20 minutes of dissolution.

### Example 5

### In Vivo Studies; Fasting

Preparation of Pseudoephedrine HCl extended-release tablet cores, 240 mg: Extended-release pseudoephedrine HCl tablet cores (drug core and extended-release coating) are prepared having the components listed in Table 9 below.

TABLE 9

| | |
|---|---|
| Component | milligram/tablet |

| Core Formulation | E | F |
|---|---|---|
| Pseudoephedrine HCl | 240 | 240 |
| Carnauba Wax, NF | 134 | 134 |
| Stearic Acid, NF | 90 | 90 |
| Denatured Alcohol | * | * |
| Hydroxy Propylcellulose, NF | 91 | 91 |
| (Klucel ® HXF) | | |
| Silicon Dioxide, NF | 5 | 5 |
| (Syloid ® 244 FP) | | |
| Magnesium Stearate, NF | 5 | 5 |
| Core coating | | |
| Surelease ® (25% w/w | 33.43 | 63.2 |
| ethylcellulose) | | |
| Opadry ® Clear # YS-3-7011 | 5.57 | 15.8 |
| Purified Water, USP | * | * |

The tablet cores were prepared according to Example 1 above. The compressed tablets are coated with a core coating prepared from a suspension of Surelease, Opadry Clear, and water to form extended-release pseudoephedrine tablet cores.

A study was performed in healthy volunteers to measure the AUC (0-24 hours and O—INF) and Cₘₐₓ following single oral doses, in the fasted state, of Formulation E containing 240 mg of pseudoephedrine hydrochloride in an extended-release tablet. The study was performed on 12 subjects. After administration of the doses, blood samples were taken from the subjects every ⅓ of an hour for the first hour, then every half hour up to three hours, then every hour up to 10 hours, and finally at hours 12, 16, 24, 36, and 48. The results were calculated as Ln-transformed data, geometric mean, as well as the least squares mean, non-transformed data. The geometric means are based on least squares means of ln-transformed values. The results are provided in Table 10 below. A similar experiment is performed for Formulation F and the results are provided in Table 11 below.

TABLE 10
Formulation E, Fasting

| | | | | |
|---|---|---|---|---|
| | | | | 90% Confidence |
| | | Reference | | Interval |
| | Formulation | [ALLEGRA-D ® 24 | % | (Lower Limit, |
| PK variable | E | HOUR] | Ratio | Upper Limit) |

Ln-transformed data

| | Geometric Mean | | | |
|---|---|---|---|---|
| Cₘₐₓ | 390.20 | 370.26 | 105.39 | (94.9, 117.03) |
| (ng/ml) | | | | |
| AUC₀₋ₜ | 7675.88 | 7202.20 | 106.58 | (97.92, 116.01) |
| (ng-hr/ml) | | | | |
| AUC_{0-INF} | 8064.90 | 7806.00 | 103.32 | (95.43, 111.86) |
| (ng-hr/ml) | | | | |

Non-transformed data

| | least squares mean | | | |
|---|---|---|---|---|
| Cmax | 414.65 | 385.34 | 107.61 | (91.88, 123.33) |
| (ng/ml) | | | | |
| AUC₀₋ₜ | 8124.09 | 7800.28 | 104.15 | (96.14, 112.16) |
| (ng-hr/ml) | | | | |
| AUC_{0-INF} | 8517.43 | 8330.10 | 102.25 | (94.64, 109.86) |
| (ng-hr/ml) | | | | |
| Tmax | 7.75 | 10.43 | 74.34 | (63.22, 85.46) |
| kₑₗᵢₘ | 0.0901 | 0.0995 | 90.57 | (77.6, 103.55) |
| t_{1/2} | 8.16 | 7.11 | 114.76 | (98.74, 130.78) |

TABLE 11
Formulation F, Fasting

| | | | | |
|---|---|---|---|---|
| | | | | 90% Confidence |
| | | Reference | | Interval |
| | Formulation | [ALLEGRA-D ® 24 | % | (Lower Limit, |
| PK variable | F | HOUR] | Ratio | Upper Limit) |

Ln-transformed data

| | Geometric Mean | | | |
|---|---|---|---|---|
| Cₘₐₓ | 441.73 | 370.26 | 119.30 | (107.43, 132.49) |
| (ng/ml) | | | | |
| AUC₀₋ₜ | 8145.07 | 7202.20 | 113.09 | (103.9, 123.1) |
| (ng-hr/ml) | | | | |
| AUC_{0-INF} | 8485.98 | 7806.00 | 108.71 | (100.41, 117.7) |
| (ng-hr/ml) | | | | |

Non-transformed data

| | least squares mean | | | |
|---|---|---|---|---|
| Cmax | 460.40 | 385.34 | 119.48 | (103.75, 135.21) |
| (ng/ml) | | | | |
| AUC₀₋ₜ | 8583.92 | 7800.28 | 110.05 | (102.04, 118.05) |
| (ng-hr/ml) | | | | |
| AUC_{0-INF} | 8918.92 | 8330.10 | 107.07 | (99.46, 114.68) |
| (ng-hr/ml) | | | | |
| Tmax | 8.00 | 10.43 | 76.74 | (65.62, 87.86) |
| kₑₗᵢₘ | 0.0947 | 0.0995 | 95.17 | (82.2, 108.15) |
| t_{1/2} | 7.64 | 7.11 | 107.48 | (91.46, 123.5) |

As the results in Table 10 indicate, under fasting conditions, Formulation E provides pharmacokinetic parameters substantially similar to the reference product, for natural log transformed geometric mean AUC values (AUC_{0-∞} and AUC_{0-∞}INF) and Cₘₐₓ. Formulation E is substantially bioequivalent to the reference product when dosed under fasting conditions (90% confidence interval of 80-125% for AUC and Cₘₐₓ are achieved by Formulation E). Formulation F is substantially bioequivalent to the reference product when dosed under fasting conditions (90% confidence interval of 80-125% for AUC is achieved by Formulation F when compared to the reference, and just outside the range for Cₘₐₓ).

### Example 6

### In Vivo Studies; Non-Fasting

A study was performed in healthy volunteers to measure the AUC (0-24 hours and 0-INF) and Cₘₐₓ following single oral doses, in the non-fasted state, of Formulation E containing 240 mg of pseudoephedrine hydrochloride in an extended-release tablet. The study was performed on 12 subjects. The subjects were provided a high fat breakfast beginning at 30 minutes before dosing. After administration of the doses, blood samples were taken from the subjects every ⅓ of an hour for the first hour, then every half hour up to three hours, then every hour up to 10 hours, and finally at hours 12, 16, 24, 36, and 48. The results were calculated as Ln-transformed data, geometric mean, as well as the least squares mean, non-transformed data. The geometric means are based on least squares means of ln-transformed values. The results are provided in Table 12 below. A similar experiment is performed for Formulation F and the results are provided in Table 13 below.

TABLE 12
Formulation E, Non-Fasting

| | | | | |
|---|---|---|---|---|
| | | | | 90% Confidence |
| | | Reference | | Interval |
| | Formulation | [ALLEGRA-D ® 24 | % | (Lower Limit, |
| PK variable | E | HOUR] | Ratio | Upper Limit) |

Ln-transformed data

| | Geometric Mean | | | |
|---|---|---|---|---|
| Cₘₐₓ | 335.96 | 378.07 | 88.86 | (74.92, 105.4) |
| (ng/ml) | | | | |
| AUC₀₋ₜ | 7176.78 | 7484.91 | 95.88 | (86.64, 106.11) |
| (ng-hr/ml) | | | | |
| AUC_{0-INF} | 7904.84 | 8179.51 | 96.64 | (89.4, 104.48) |
| (ng-hr/ml) | | | | |

Non-transformed data

| | least squares mean | | | |
|---|---|---|---|---|
| Cmax | 396.66 | 395.48 | 100.30 | (86.88, 113.72) |
| (ng/ml) | | | | |
| AUC₀₋ₜ | 7866.57 | 7919.18 | 99.34 | (91.63, 107.04) |
| (ng-hr/ml) | | | | |
| AUC_{0-INF} | 8466.72 | 8660.77 | 97.76 | (91.95, 103.57) |
| (ng-hr/ml) | | | | |
| Tmax | 9.83 | 11.67 | 84.29 | (71.3, 97.27) |
| kₑₗᵢₘ | 0.0751 | 0.0833 | 90.21 | (74.67, 105.74) |
| t_{1/2} | 10.95 | 8.64 | 126.73 | (100.23, 153.24) |

TABLE 13
Formulation F, Non-Fasting

| | | | | |
|---|---|---|---|---|
| | | | | 90% Confidence |
| | | Reference | | Interval |
| | Formulation | [ALLEGRA-D ® 24 | % | (Lower Limit, |
| PK variable | F | HOUR] | Ratio | Upper Limit) |

Ln-transformed data

| | Geometric Mean | | | |
|---|---|---|---|---|
| Cₘₐₓ | 456.76 | 378.07 | 120.82 | (101.86, 143.3) |
| (ng/ml) | | | | |
| AUC₀₋ₜ | 8658.35 | 7484.91 | 115.68 | (104.53, 128.01) |
| (ng-hr/ml) | | | | |
| AUC_{0-INF} | 8994.71 | 8179.51 | 109.97 | (101.72, 118.88) |
| (ng-hr/ml) | | | | |

Non-transformed data

| | least squares mean | | | |
|---|---|---|---|---|
| Cmax | 487.92 | 395.48 | 123.38 | (109.95, 136.8) |
| (ng/ml) | | | | |
| AUC₀₋ₜ | 9166.05 | 7919.18 | 115.75 | (108.04, 123.45) |
| (ng-hr/ml) | | | | |
| AUC_{0-INF} | 9478.54 | 8660.77 | 109.44 | (103.63, 115.25) |
| (ng-hr/ml) | | | | |
| Tmax | 11.42 | 11.67 | 97.86 | (84.87, 110.84) |
| kₑₗᵢₘ | 0.1035 | 0.0833 | 124.30 | (108.76, 139.83) |
| t_{1/2} | 6.95 | 8.64 | 80.45 | (53.94, 106.96) |

As the results in Table 12 indicate, under non-fasting conditions, Formulation E provides pharmacokinetic parameters substantially similar to the reference product, particularly for natural log transformed geometric mean AUC values (AUC₀₋ₜ and AUC_{0-∞}INF). Formulation E is substantially bioequivalent to the reference product when dosed under non-fasting conditions (90% confidence interval of 80-125% for AUC is achieved by Formulation E when compared to the reference, and just outside the range for Cₘₐₓ).

### Example 7

### Dissolution of Exemplary Fexofenadine Immediate Release Formulations

To determine the effect of the various fexofenadine hydrochloride polymorphs on dissolution of fexofenadine formulations, different forms of fexofenadine hydrochloride were compressed into immediate-release tablets according to Table 14. The various polymorphic forms tested are given in Table 15.

TABLE 14

| Ingredient | G | H | I | J | K | L | M |
|---|---|---|---|---|---|---|---|
| Fexofenadine HCl | 180 | 180 | 180 | 180 | 180 | 180 | 180 |
| Avicel ® pH 102 | 78 | 78 | 78 | 78 | 78 | 78 | 78 |
| Ac-Di-Sol ® | 20 | 20 | 20 | 20 | 20 | 20 | 20 |
| Starch 1500 ® | 20 | 20 | 20 | 20 | 20 | 20 | 20 |
| Magnesium sulfate | 2 | 2 | 2 | 2 | 2 | 2 | 2 |
| Total | 300 | 300 | 300 | 300 | 300 | 300 | 300 |

TABLE 15

| Tablet Code | Polymorphic form |
|---|---|
| G | Form 16 and 20 mix, unmilled |
| H | Form 16 and Form 20 mix, milled |
| I | Form I |
| J | Form X |
| K | Polymorphic form unknown |
| L | Amorphous |
| M | Form C |

Tablets A-G were tested according to USP 26, 711 using 900 milliliters of a dissolution medium at 37° C.±0.5° C. and a paddle speed of 50 rotations per minute (rpm) in 0.1 N HCl or pH 4.5 acetate buffer. The results are given in Tables 16 and 17 and compared to ALLEGRA D® and ALLEGRA® 180 mg.

TABLE 16
0.1 N HCl

| Time, | | | | | | | | ALLEGRA | ALLEGRA ® |
|---|---|---|---|---|---|---|---|---|---|
| Min | G | H | I | J | K | L | M | D ® 24 | 180 mg |
| 0 | 0 | 0 | 0 | 0 | 0 | 0 | 0 | 0 | 0 |
| 5 | 14 | 6 | 87 | 51 | 23 | 2 | 83 | 24 | 68 |
| 10 | 21 | 10 | 93 | 68 | 29 | 2 | 93 | 68 | 87 |
| 15 | 26 | 13 | 97 | 77 | 33 | 3 | 97 | 96 | 93 |
| 20 | 30 | 16 | 98 | 84 | 36 | 4 | 99 | 101 | 96 |
| 25 | 33 | 18 | 100 | 89 | 39 | 4 | 99 | 102 | 98 |
| 30 | 36 | 20 | 100 | 91 | 41 | 5 | 100 | 102 | 99 |
| 45 | 42 | 25 | 99 | 96 | 46 | 6 | 100 | 101 | 101 |
| 60 | 47 | 29 | 101 | 98 | 50 | 7 | 101 | 101 | 101 |
| 90 | 55 | 36 | 101 | 100 | 57 | 9 | 101 | 101 | 102 |
| 120 | 61 | 41 | 101 | 101 | 62 | 11 | 101 | 100 | 102 |

TABLE 17
pH 4.5 acetate buffer

| Time, | | | | | | | | ALLEGRA | ALLEGRA ® |
|---|---|---|---|---|---|---|---|---|---|
| Min | G | H | I | J | K | L | M | D ® 24 | 180 mg |
| 0 | 0 | 0 | 0 | 0 | 0 | 0 | 0 | 0 | 0 |
| 5 | 16 | 2 | 84 | 67 | 32 | 1 | 73 | 5 | 28 |
| 10 | 21 | 3 | 92 | 73 | 37 | 2 | 77 | 24 | 38 |
| 15 | 23 | 4 | 93 | 74 | 39 | 2 | 79 | 47 | 43 |
| 20 | 24 | 4 | 93 | 75 | 41 | 3 | 80 | 63 | 47 |
| 25 | 26 | 4 | 93 | 76 | 42 | 3 | 81 | 71 | 50 |
| 30 | 26 | 5 | 94 | 77 | 43 | 3 | 81 | 75 | 53 |
| 45 | 29 | 6 | 94 | 78 | 46 | 4 | 82 | 80 | 58 |
| 60 | 31 | 7 | 94 | 79 | 48 | 4 | 83 | 82 | 62 |
| 90 | 34 | 9 | 94 | 81 | 52 | 4 | 85 | 84 | 68 |
| 120 | 36 | 11 | 94 | 82 | 55 | 5 | 86 | 85 | 72 |

As can be seen from Tables 16 and 17, fexofenadine hydrochloride Form C (Tablet M) and fexofenadine hydrochloride Form X (Tablet J) have a similar immediate-release profile to fexofenadine hydrochloride Form I (Tablet I, ALLEGRA D® and ALLEGRA® 180 mg).

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-15. (canceled)

16. A controlled-release formulation, comprising:
a tablet core comprising pseudoephedrine or a pharmaceutically acceptable salt thereof, and a wax excipient; and
an extended-release coating substantially surrounding the tablet core comprising a release-retarding coating material; and
an immediate-release portion comprising an antihistamine as an immediate-release active agent;
wherein the formulation exhibits substantially no food effect.

17. The formulation of claim 16, wherein the wax excipient is a wax having a melting temperature greater than 20° C.

18. The formulation of claim 16, wherein the wax excipient is carnauba wax, vegetable wax, fruit wax, microcrystalline wax, bees wax, hydrocarbon wax, paraffin wax, cetyl esters wax, non-ionic emulsifying wax, anionic emulsifying wax, candelilla wax, stearyl alcohol, cetyl alcohol, cetostearyl alcohol, lauryl alcohol, myristyl alcohol, a hydrogenated vegetable oil, a hydrogenated castor oil, a fatty acid, a fatty acid ester, a fatty acid glyceride, a polyethylene glycol having a Mₙ of greater than about 3000, or a combination comprising at least one of the foregoing wax excipients.

19. The formulation of claim 16, wherein the wax excipient is present in an amount of about 5 to about 60 wt. % based on the total weight of the core.

20. The formulation of claim 16, wherein the core further comprises an additional release-retarding material, wherein the additional release-retarding material is an acrylic polymer, an alkylcellulose, a substituted alkylcellulose, shellac, zein, polyvinylpyrrolidine, crosslinked polyvinylpyrrolidinone, a vinyl acetate copolymer, polyethylene oxide, a polyvinyl alcohol, or a combination comprising at least one of the foregoing additional release-retarding materials.

21. (canceled)

22. The formulation of claim 16, wherein the release-retarding coating material comprises a film forming polymer, wherein the film forming polymer is an alkylcellulose, a hydroxyalkylcellulose, a hydroxyalkyl alkylcellulose, a carboxyalkylcellulose, an alkali metal salt of a carboxyalkylcellulose, a carboxyalkyl alkylcellulose, a carboxyalkylcellulose ester, a starch, a pectin, a chitine derivate, a polysaccharide, a carrageenan, a galactomannas, traganth, agar-agar, gum arabicum, guar gum, xanthan gum, a polyacrylic acid, a polymethacrylic acid, a methacrylate copolymer, a polyvinylalcohol, polyvinylpyrrolidone, a copolymer of polyvinylpyrrolidone with vinyl acetate, a polyalkylene oxide, a copolymer of ethylene oxide and propylene oxide, or a combination comprising at least one of the foregoing film forming polymers.

23. The formulation of claim 22, wherein the film forming polymer is methylcellulose, ethylcellulose, hydroxymethyl cellulose, hydroxyethylcellulose, hydroxypropylcellulose, hydroxybutylcellulose, hydroxyethyl methylcellulose, hydroxypropyl methylcellulose, carboxymethylcellulose, sodium carboxymethylcellulose, carboxymethyl ethylcellulose, carboxymethylcellulose ester, sodium carboxymethylamylopectine, chitosan, alginic acid, alkali metal salt of alginic acid, ammonium salt of alginic acid, or a combination comprising at least one of the foregoing film forming polymers.

24. The formulation of claim 16, wherein the extended-release coating is present at about 0.1 to about 30 wt. % based on the total weight of the core and extended-release coating.

25. The formulation of claim 16, wherein the extended-release coating is present at about 5.0 to about 20 wt. % based on the total weight of the core and extended-release coating.

26. The formulation of claim 16, wherein the immediate-release portion is in the form of a layer disposed on at least a portion of the core, or
wherein the immediate-release portion is in the form of a layer disposed on at least a portion of the core.

27. (canceled)

28. The formulation of claim 16, wherein the core comprises pseudoephedrine or a pharmaceutically acceptable salt thereof, carnauba wax, and hydroxypropyl cellulose;
wherein the extended-release coating comprises ethyl cellulose and hydroxypropyl methylcellulose; and
wherein the immediate-release portion comprises fexofenadine or a pharmaceutically acceptable salt thereof.

29. The formulation of claim 28, wherein the formulation provides a Tₘₐₓ of pseudoephedrine at about 11 to about 13 hours; and a Tₘₐₓ of fexofenadine at about 1.6 to about 2.2 hours.

30. The formulation of claim 28, wherein after a single administration of the formulation, the formulation provides a Cₘₐₓ of pseudoephedrine of about 360 ng/mL to about 430 ng/mL; and a Cₘₐₓ of fexofenadine of about 600 ng/mL to about 660 ng/mL; or
wherein after administration of five or more doses of the formulation the formulation provides a Cₘₐₓ of pseudoephedrine of about 450 ng/mL to about 550 ng/mL; and a Cₘₐₓ of fexofenadine of about 640 ng/mL to about 710 mg/mL.

31. (canceled)

32. A controlled-release formulation, comprising:
a tablet core comprising pseudoephedrine or a pharmaceutically acceptable salt thereof, and a wax excipient;
an extended-release coating substantially surrounding the tablet core comprising a release-retarding coating material; and
an immediate-release portion comprising fexofenadine or a pharmaceutically acceptable salt thereof
wherein the formulation is bioequivalent to a reference drug product according to New Drug Application No. 021704.

33. The formulation of claim 32, wherein the formulation exhibits a ratio of a geometric mean of logarithmic transformed AUC₀₋ₜ or AUC_{0-∞} of the composition to a geometric mean of logarithmic transformed AUC₀₋ₜ or AUC_{0-∞} of a reference drug product according to New Drug Application No. 021704 of about 0.80 to about 1.25; wherein the 90% confidence limits of the ratio are about 0.80 to about 1.25.

34. (canceled)

35. The formulation of claim 32, wherein the formulation exhibits a ratio of a geometric mean of logarithmic transformed Cₘₐₓ of the formulation to a geometric mean of logarithmic transformed Cₘₐₓ of a reference drug product according to New Drug Application No. 021704 of about 0.80 to about 1.25; wherein the 90% confidence limits of the ratio are about 0.80 to about 1.25.

36. (canceled)

37. The formulation of claim 32, wherein the formulation exhibits a ratio of a geometric mean of logarithmic transformed Cₘₐₓ of the formulation to a geometric mean of logarithmic transformed Cₘₐₓ of a reference drug product according to New Drug Application No. 021704 of about 0.70 to about 1.43.

38. The formulation of claim 32, wherein the bioequivalence was determined under fasting conditions; or
wherein the bioequivalence was determined under non-fasting conditions.

39. (canceled)

40. The formulation of claim 32, wherein the dissolution profile of the formulation is substantially the same as the dissolution profile of an equivalent strength of a reference drug product according to New Drug Application No. 021704 when tested under the conditions according to USP 28<711> test method 2 (paddle), using of 900 ml of purified water at 37° C.±0.5° C., and 50 rpm paddle speed.

41. A controlled-release formulation, comprising:
a tablet core comprising pseudoephedrine or a pharmaceutically acceptable salt thereof, and a wax excipient; and
an extended-release coating substantially surrounding the tablet core comprising a release-retarding coating material;
wherein the formulation exhibits a dissolution profile such that at five hours after combining the formulation with 900 ml of a dissolution medium at 37° C.±0.5° C. according to USP 28<711> test method 2 (paddle), 50 rpm paddle speed, about 25 to about 50 wt. % of the total amount of active agent is released;
wherein the dissolution medium is purified water, 0. N HCl, pH 4.5 acetate buffer, or pH 6.8 phosphate buffer.

42. The formulation of claim 41, wherein after three hours, about 15 to about 35 wt. % of the total amount of the pseudoephedrine or pharmaceutically acceptable salt thereof is released.

43. The formulation of claim 41, wherein after seven hours, about 35 to about 65 wt. % of the total amount of the pseudoephedrine or pharmaceutically acceptable salt thereof is released.

44. The formulation of claim 41, wherein after 9 hours greater than or equal to about 45% of the total amount of the pseudoephedrine or pharmaceutically acceptable salt thereof is released.

45. (canceled)

46. The controlled-release formulation of claim 16,
wherein the formulation exhibits a dissolution profile after combining the formulation with 900 ml of purified water at 37° C.±0.5° C. according to USP 28<711> test method 2 (paddle), 50 rpm paddle speed,
wherein about 13 to about 40 wt. % of the total amount of the pseudoephedrine or a pharmaceutically acceptable salt thereof is released after three hours;
about 20 to about 60 wt. % of the total amount of the pseudoephedrine or a pharmaceutically acceptable salt thereof is released after five hours; and
about 40 to about 80 wt. % of the total amount of the pseudoephedrine or a pharmaceutically acceptable salt thereof is released after seven hours.

47. The controlled-release formulation of claim 16,
wherein the formulation exhibits a dissolution profile after combining the formulation with 900 ml of a dissolution medium at 37° C.±0.5° C. according to USP 28 <711> test method 2 (paddle), 50 rpm paddle speed,
wherein about 15 to about 35 wt. % of the total amount of the pseudoephedrine or a pharmaceutically acceptable salt thereof core active agent is released after three hours;
about 25 to about 50 wt. % of the total amount of the pseudoephedrine or a pharmaceutically acceptable salt thereof is released after five hours;
about 35 to about 65 wt. % of the total amount of the pseudoephedrine or a pharmaceutically acceptable salt thereof is released after seven hours; and
about 45 to about 75 wt. % of the total amount of the pseudoephedrine or a pharmaceutically acceptable salt thereof is released after nine hours;
wherein the dissolution medium is purified water 0.1N HCl, pH 4.5 acetate buffer, or pH 6.8 phosphate buffer.

48.-51. (canceled)
